(19) United States
(12) Patent Application Publication
(10) Pub. No.: US 2004/0068224 A1
(43) Pub. Date: Apr. 8, 2004

(54) Electroactive polymer actuated medication infusion pumps

(76) Inventors: Lucien Alfred Couvillon JR., Concord, MA (US); Pete M. Nicholas, Boston, MA (US); Michael S. Banik, Boston, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/262,991

(22) Filed: Oct. 2, 2002

(51) Int. Cl. A61M 31/00

(52) U.S. Cl. 604/67; 604/133; 604/891.1

(57) ABSTRACT

The present invention is directed to a drug delivery pump apparatus, which comprises: (a) an expandable and contractible enclosure having an interior volume that defines a medication reservoir; (b) one or more electroactive polymer actuators; (c) a medication outlet port providing fluid communication between the interior volume of the contractible and expandable enclosure and an exterior of the delivery pump apparatus; and (d) a control unit electrically coupled to the one or more actuators and sending control signals to the same. The one or more electroactive polymer actuators act to reduce the interior volume of the contractible and expandable enclosure based upon the received control signals. The present invention is also directed to a method of delivering a liquid therapeutic agent to a patient. The method comprises: (a) providing the above infusion pump apparatus; (b) placing the outlet port of the infusion pump apparatus in fluid communication with a patient; and (c) sending the control signals to the one or more actuators to reduce the internal volume of the contractible and expandable enclosure, thereby forcing a portion of the liquid therapeutic agent within the medication reservoir through the outlet port and into the patient.

### FIELD OF THE INVENTION

The present invention relates to medication infusion pumps and more particularly to medication infusion pumps that are driven by electroactive polymer actuators.

### BACKGROUND OF THE INVENTION

Infusion pumps are known in which a selected medication is delivered to a patient in accordance with a constant, patient-controlled, sensor-controlled or programmable administration schedule. Numerous therapeutic applications have been proposed for such pumps, including nitroglycerine for coronary vascular spasm, insulin for diabetes, theophylline for asthma, antineoplastic agents (for example, floxuridine) for the treatment of cancer, lidocaine for cardiac arrhythmia, antimicrobial and antiviral agents for chronic infection (e.g. osteomyelitis), morphine and other opiates, endorphines and analgesics for chronic intractable pain.

In recent years, infusion pumps have been developed for direct implantation into the body of a patient, allowing medication to be delivered to the patient in controlled doses over an extended period of time. Examples of infusion pumps can be found, for example, in U.S. Pat. No. 3,731,681, U.S. Pat. No. 4,468,220, U.S. Pat. No. 4,718,893, U.S. Pat. No. 4,813,951, U.S. Pat. No. 4,573,994, U.S. Pat. No. 5,820,589, U.S. Pat. No. 5,957,890 and U.S. Pat. No. 6,203,523, which are incorporated by reference in their entireties. Such implantable infusion pumps typically include an internal medication reservoir for receiving, storing and dispensing a selected medication, in liquid form, to a patient. Medication may be dispensed to an intended destination organ through a catheter that is attached to the infusion pump, with the catheter being used to accesses the blood flow to the organ (e.g., via an artery supplying the organ). In other instances, medication is delivered via catheter to the venous system, for example, for the delivery of sedatives and or pain medication.

It is also common to provide such implantable infusion pumps with an access port, which is provided with a resealable septum. To refill the medication reservoir, a hypodermic needle is typically inserted through the septum and into a chamber between the septum and a needle stop. The medication is injected under pressure into the chamber and flows into the reservoir.

In some infusion pumps, medication is delivered from the medication reservoir into the body of the patient by a miniature pump, which is programmably controlled for delivering the medication to the patient in selected doses at selected times. Such pumps typically include a drug reservoir, a pump, such as a peristaltic pump, to pump the medication from the reservoir, and an outlet port (e.g., a catheter port) to transport the drug from the reservoir via the pump to a patient's anatomy. Such devices also typically include a battery or transdermal coupling to power the pump as well as an electronic module to control the flow rate of the pump. Some models further include a wireless transceiver to permit remote programming of the electronic module. Unfortunately, such pumps are typically bulky and energy inefficient.

In other infusion pumps, two adjacent chambers are provided which are separated, for example, by a flexible metal bellows. One chamber acts as a medication reservoir, while the other contains a propellant fluid in liquid-vapor equilibrium. The vapor pressure of the propellant fluid exerts a relatively constant pressure on the bellows, forcing the medication from the drug reservoir, through an appropriate flow restriction (e.g., an orifice or capillary tube), to an outlet port. Flow rate is typically metered by using different orifice sizes or lengths of flow-restrictive capillary tubing. Somewhat analogous to electrical current, the flow rate of the medication increases with (a) an increase in pressure, (b) an increase in the diameter of the orifice or capillary tube and (c) a decrease in the length of the capillary tube. The flow rate from such pumps is continuous and substantially constant. FIG. 1 illustrates one such infusion pump, generally designated 100, from U.S. Pat. No. 3,731,681, the entire disclosure of which is incorporated by reference. The pump 100 includes housing 110, propellant chamber 123 and medication chamber 124 separated by bellows 117, access port 139, including septum 138, capillary tube 140, and passageway 137 between access port 139 and medication chamber 124. Unfortunately, such pumps are bulky and medication flow rate is essentially constant, rather than variable.

### SUMMARY OF THE INVENTION

The present invention is directed to novel implantable infusion pumps in which electroactive polymer actuators are used to express medication from a medication reservoir within the pump.

According to a first aspect of the present invention, a drug delivery pump apparatus is provided that comprises: (a) an expandable and contractible enclosure having an interior volume that defines a medication reservoir; (b) one or more electroactive polymer actuators; (c) a medication outlet port providing fluid communication between the interior volume of the contractible and expandable enclosure and an exterior of the delivery pump apparatus; and (d) a control unit electrically coupled to the one or more actuators and sending control signals to the same. The one or more electroactive polymer actuators act to reduce or increase the interior volume of the contractible and expandable enclosure based upon the received control signals.

In some embodiments, the interior volume of the contractible and expandable enclosure is reduced upon expansive activation of the one or more electroactive polymer actuators. For example, the one or more electroactive polymer actuators can be disposed between a housing and the contractible and expandable enclosure (for instance, a bellows), such that the enclosure is compressed upon expansion of the one or more electroactive polymer actuators.

In other embodiments, the interior volume of the contractible and expandable enclosure is reduced upon contraction of the one or more electroactive polymer actuators. For example, the contractible and expandable enclosure can include an elastic bladder whose interior volume is decreased upon electroactive polymer actuator contraction. For instance, the one or more electroactive polymer actuators can be disposed within or upon the walls of the elastic bladder.

Typically, the one or more electroactive polymer actuators will comprise an electroactive polymer, a counter-electrode, and an electrolyte-containing region disposed intermediate the electroactive polymer and the counter-electrode.

According to another aspect of the present invention, a method is provided for delivering a liquid therapeutic agent to a patient. The method comprises: (a) providing the above infusion pump apparatus; (b) placing the outlet port of the infusion pump apparatus in fluid communication with a patient; and (c) sending control signals to the one or more actuators to reduce the internal volume of the contractible and expandable enclosure, thereby forcing a portion of the liquid therapeutic agent that resides within the medication reservoir through the outlet port and into the patient. In many embodiments, the infusion pump apparatus is implanted or inserted within the patient.

Control signals for the one or more actuators can be generated, for example, based on a user-activated switch (which can be inserted or implanted within the patient, if desired), based on the passage of a predetermined interval of time, based upon input from a chemical sensor that measures a detectable chemical species, and so forth.

An advantage of the present invention is that infusion pumps can be provided, which are energy efficient and volume efficient (i.e., they are compact).

The present invention is also advantageous in that infusion pumps can be provided, which are electronically controlled, allowing for precise, programmed control of the infusion of medication.

The present invention is further advantageous in that infusion pumps can be provided, which are simple and easy to manufacture.

These and other embodiments and advantages of the present invention will become apparent from the following detailed description, and the accompanying drawings, which illustrate by way of example the features of the invention.

### BRIEF DESCRIPTION OF THE DRAWINGS

FIG. 1 is a partial cross-sectional view of an infusion pump.

FIG. 2 is a schematic cross-sectional view of an electroactive polymer actuator useful in connection with certain embodiments of the present invention.

FIG. 3 is a schematic cross-sectional view of an infusion pump in accordance with an embodiment of the present invention.

FIG. 4A is a schematic cross-sectional view of an infusion pump in accordance with another embodiment of the present invention.

FIG. 4B is a schematic enlarged cross-sectional view corresponding to region A of FIG. 4A, in accordance with an embodiment of the present invention.

FIG. 5A is a schematic cross-sectional view of an infusion pump in accordance with yet another embodiment of the present invention.

FIG. 5B is a schematic enlarged cross-sectional view corresponding to region A of FIG. 5A, in accordance with an embodiment of the present invention.

FIG. 5C is a schematic enlarged cross-sectional view corresponding to region A of FIG. 5A, in accordance with an alternative embodiment of the present invention.

FIG. 6 is a schematic perspective view of an infusion pump in accordance with another embodiment of the present invention.

FIG. 7 depicts an infusion pump in block diagram format in accordance with another embodiment of the present invention.

### DETAILED DESCRIPTION OF THE INVENTION

The present invention now will be described more fully hereinafter with reference to the accompanying drawings, in which several embodiments of the present invention are shown. This invention may, however, be embodied in different forms and should not be construed as limited to the embodiments set forth herein.

According to an embodiment of the invention, an infusion pump (also referred to herein as a “drug delivery pump”) is provided in which electroactive polymer actuators are utilized to express medication from a medication reservoir within the pump. Actuators based on electroactive polymers are preferred for the practice of the present invention, for example, due to their small size, large force and strain, low cost and ease of integration into the infusion pumps of the present invention.

Electroactive polymers, members of the family of plastics referred to as “conducting polymers,” are a class of polymers characterized by their ability to change shape in response to electrical stimulation. They typically structurally feature a conjugated backbone and have the ability to increase electrical conductivity under oxidation or reduction. Some common electroactive polymers are polyaniline, polysulfone, polypyrrole and polyacetylene. Polypyrrole is pictured below:

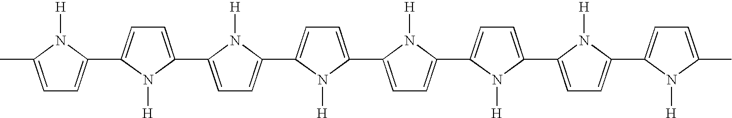

These materials are typically semi-conductors in their pure form. However, upon oxidation or reduction of the polymer, conductivity is increased. The oxidation or reduction leads to a charge imbalance that, in turn, results in a flow of ions into the material in order to balance charge. These ions, or dopants, enter the polymer from an ionically conductive electrolyte medium that is coupled to the polymer surface. The electrolyte may be, for example, a gel, a solid, or a liquid. If ions are already present in the polymer when it is oxidized or reduced, they may exit the polymer.

It is well known that dimensional changes may be effectuated in certain conducting polymers by the mass transfer of ions into or out of the polymer. For example, in some conducting polymers, expansion is due to ion insertion between chains, whereas in others inter-chain repulsion is the dominant effect. Regardless of the mechanism, the mass transfer of ions into and out of the material leads to an expansion or contraction of the polymer.

Currently, linear and volumetric dimensional changes on the order of 25% are possible. The stress arising from the dimensional change can be on the order of 3 MPa, far exceeding that exerted by smooth muscle cells, allowing substantial forces to be exerted by actuators having very small cross-sections. These characteristics are ideal for construction of the infusion pumps of the present invention.

Referring now to FIG. 2, an electroactive polymer actuator 10 is shown schematically in cross-section. Active member 12 of actuator 10 has a surface coupled with electrolyte 14 and has an axis 11. Active member 12 includes an electroactive polymer that contracts or expands in response to the flow of ions out of, or into, the active member 12. Ions are provided by electrolyte 14, which adjoins member 12 over at least a portion, and up to the entirety, of the surface of active member 12 in order to allow for the flow of ions between the two media.

Many geometries are available for the relative disposition of member 12 and electrolyte 14. In accordance with some embodiments of the invention, member 12 may be a film, a fiber or a group of fibers, or a combination of multiple films and fibers disposed so as to act collectively to apply a tensile force in a longitudinal direction substantially along axis 11 in this instance. The fibers may be bundled or distributed within the electrolyte 14.

Active member 12 includes an electroactive polymer. Many electroactive polymers having desirable tensile properties are known to persons of ordinary skill in the art. In accordance with some embodiments of the invention, active member 12 can be a polypyrrole film. Such a polypyrrole film may be synthesized, for example, by electrodeposition according to the method described by M. Yamaura et al., “Enhancement of Electrical Conductivity of Polypyrrole Film by Stretching: Counter-ion Effect,” Synthetic Metals, vol. 36, pp. 209-224 (1988), which is incorporated herein by reference. In addition to polypyrrole, any conducting polymer that exhibits contractile or expansile properties may be used within the scope of the invention. Polyaniline, polysulfone, polyacetylene are examples.

Electrolyte 14 may be, for example, a liquid, a gel, or a solid, so long as ion movement is allowed. Moreover, where the electrolyte 14 is a solid, it will typically move with the active member 12 and will typically not be subject to delamination. Where the electrolyte 14 is a gel, it may be, for example, an agar or polymethylmethacrylate (PMMA) gel containing a salt dopant. Where the electrolyte is a liquid, it may be, for example, a phosphate buffer solution, KCl, NaCl and so forth. The electrolyte may be non-toxic in the event that a leak inadvertently occurs in vivo.

Counter electrode 18 is in electrical contact with electrolyte 14 in order to provide a return path for charge to a source 20 of potential difference between member 12 and electrolyte 14. Counter electrode 18 may be any suitable electrical conductor, for example, another conducting polymer, a conducting polymer gel, or a metal such as gold or platinum, which can be, for example, in wire or film form and can be applied, for example, by electroplating, chemical deposition, or printing. In order to activate actuator 10, a current is passed between active member 12 and counter electrode 18, inducing contraction or expansion of member 12. Additionally, the actuator may have a flexible skin for separating the electrolyte from an ambient environment.

The actuator can be provided in an essentially infinite array of configurations as desired, including planar actuator configurations (e.g., with planar active members and counter-electrodes), cylindrical actuator configurations (e.g., see the actuator illustrated in FIG. 2, which is illustrated as having a cylindrical active member and wire coil counter electrode), and so forth.

Additional information regarding the construction of actuators, their design considerations, and the materials and components that may be employed therein, can be found, for example, in U.S. Pat. No. 6,249,076, assigned to Massachusetts Institute of Technology, and in Proceedings of the SPIE, Vol. 4329 (2001) entitled “Smart Structures and Materials 2001: Electroactive Polymer and Actuator Devices (see, in particular, Madden et al, “Polypyrrole actuators: modeling and performance,” at pp. 72-83), both of which are hereby incorporated by reference in their entirety.

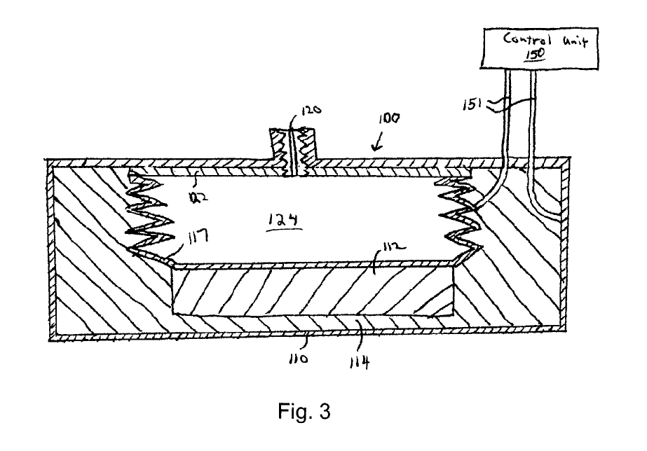

One or more electroactive polymer actuators can be disposed within the infusion pumps of the present invention in a wide variety of configurations. For example, referring now to FIG. 3, an implantable infusion pump, generally designated by the numeral 100, is illustrated in accordance with an embodiment of the present invention. The infusion pump 100 is provided with an outer housing 110. Within housing 110 is provided a bellows 117, which defines a medication reservoir 124.

An outlet port 120 provides fluid communication between the medication reservoir 124 and the exterior of the device. The outlet port 120 may be of sufficiently small diameter to ensure that, at most, insignificant amounts of medication flow from the pump when it is not driven by the actuators (this function can also be provided, at least in part, by an attached delivery catheter).

The outlet port 120 can also be provided with one or more valves (not shown). For example, a check valve can be provided to prevent back-flow of material into the pump. Check valves are valves that allow fluid to flow in a one direction, while closing to prevent backflow in the opposite direction. Examples include duckbill check valves, poppet check valves, umbrella check valves, swing check valves, tilting disk check valves, spring loaded check valves, leaflet valves and wafer check valves.

Alternatively, the outlet port can be provided with an electrically controlled valve or regulating orifice (not shown), which can be operated by the same control unit that is used to operate the electroactive polymer actuator(s) in the pump. Control valves are available based on a number of actuated valving elements, for example, ball, cone, sleeve, poppet, rotary spool or sliding spool valve elements. In other embodiments, the regulating orifice of the valve can itself be constructed with electroactive polymer actuators to provide an additional degree of control of medication delivery pressure, rate or volume. These valves can be used, for example, when reservoir vacuum is used to sample blood as well as to replenish medication. For instance, the valve can be disposed between the reservoir and the outlet port and can be held in the closed position during medication replenishment and in the open position during blood sampling.

Between the bellows 117 and the housing 110 of the infusion pump 100 of FIG. 3 are provided an active region 112 and an electrolyte-containing region 114. In this particular embodiment, the housing 110 serves as a counter-electrode to the actuator while the bellows 117 provides electrical contact with the active region. Hence, the bellows 117 and housing 110 are conductive, typically metallic, in this embodiment. In the case where the infusion pump 100 is to be implanted or inserted within a patient, the housing 110 can be, for example, a relatively inert metal such as titanium or, alternatively, a passivated metal. Of course, a non-biocompatible material can also be used for the housing 110, for example, where an additional outer layer of a biocompatible material is provided to prevent exposure of the housing material to the body.

As previously discussed, the active region 112 preferably comprises an electroactive polymer, many of which are known in the art. Polypyrrole, polysulfone, polyacetylene and polyaniline are specific examples.

The electrolyte within the electrolyte-containing region 114 can be, for example, a liquid, a gel, or a solid as previously discussed. To prevent short-circuiting, it is beneficial that the active region 112 avoid contact with the counter-electrode (i.e., the housing 110 in this embodiment). The characteristics of the electrolyte that is selected may inherently prevent such contact from occurring, particularly in the case of a solid electrolyte. If not, for example, where a liquid or non-robust gel is used as an electrolyte, additional measures may be taken to keep the active region 112 separated from the counter-electrode (housing 110 in this instance). As a specific example, a series of insulating material spacers with interstitial electrolyte can be placed between the active region 112 and the housing 110 in areas where contact is a potential problem. Similarly the electrolyte may be provided within pores or perforations of an insulating material layer or within the interstices of a woven layer or mesh of insulating material to prevent short-circuiting. Several insulating polymeric materials are listed below. PTFE is one specific example.

In this embodiment, an insulating layer 122 (which is made of any electrically insulating material, for example, one of the insulating polymers described below) is provided between the bellows 117 and the housing 110 to prevent contact between the same.

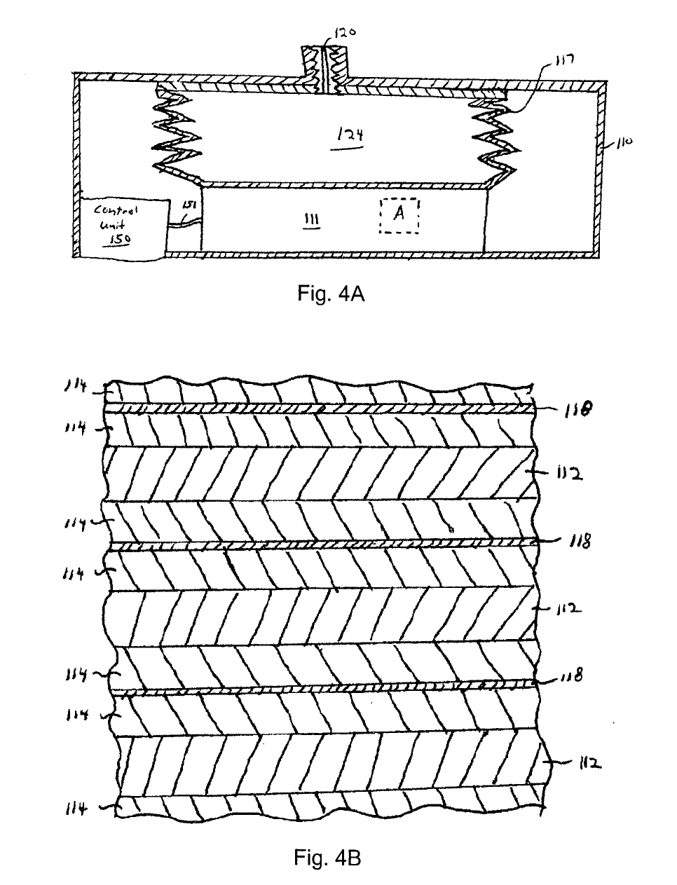

The bellows 117 and the housing 110 of the infusion pump 100 are placed in electrical connection with a control unit 150, for example, by means of insulated electrical wires 151. (Alternatively, one of the electrical wires 151 can be attached directly to the active region 112, with analogous results being achieved due to the conductivity of the of the active region 112.) An electrical potential is applied across the bellows 117 and housing 110 using the control unit 150. So long as this electrical potential is of sufficient magnitude and polarity, it will cause the active region 112 to swell, which in turn will compress the bellows 117, pressurizing the medication in the medication reservoir 124, and forcing it through the outlet port 120. A catheter is typically attached to the outlet port 120 of the infusion pump 100 to direct the medication to a desired site within the body of a patient, as is well known in the art. Although provided outside the pump housing 110 in this embodiment, the control unit can also be provided within housing 110 where desired (see, e.g., FIG. 4A below).

The energy efficiency of the electroactive polymer infusion pumps of the present invention can be enhanced by employing electroactive polymers that have inherent latching properties. By “latching property” is meant the property wherein the electroactive polymer maintains its shape (e.g., its degree of expansion), even after interruption of the electrical potential applied to expand the electroactive polymer.

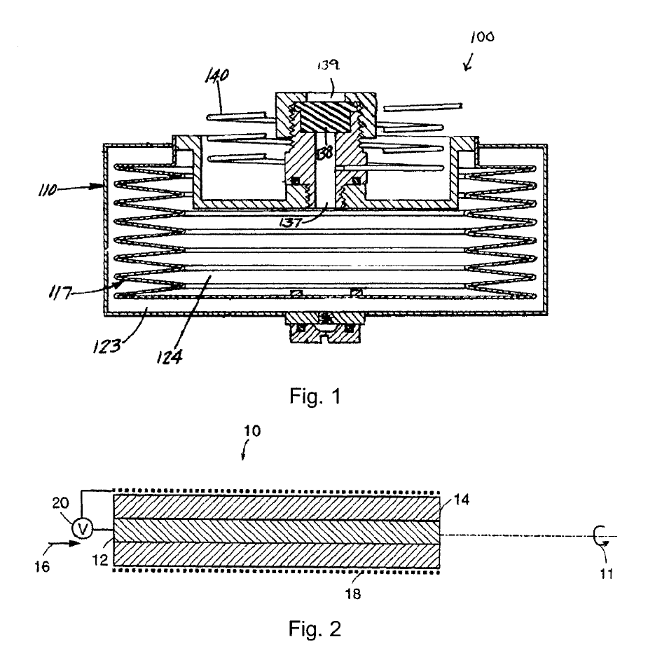

The pump of FIG. 3 (and indeed all infusion pumps described herein) may be provided with numerous features of presently known infusion pumps. As a specific example, the infusion pumps of the present invention can be equipped with an access port to recharge the pump with medication (see, e.g., FIG. 1 above). To recharge the medication reservoir, a hypodermic needle may be inserted through a septum and into a chamber between the septum and a needle stop. The medication is injected under pressure into the chamber and flows into the medication reservoir. At the same or an earlier time, an appropriate electrical potential (typically having a polarity opposite that used to contract the medication reservoir) may be applied to the actuator to create a vacuum within the reservoir for the medication, drawing in the replenishing medication.

This phenomenon can also be used to periodically analyze blood or other bodily fluid that is accessed by the catheter by drawing the bodily fluid into the device. For this purpose, a sensor (not illustrated) can be disposed, for example, within the reservoir or within catheter body.

An infusion pump in accordance with another embodiment of the present invention is illustrated in FIG. 4A. As in FIG. 3, the infusion pump 100 contains a bellows 117, which defines a medication reservoir 124. An outlet port 120 provides fluid communication between the medication reservoir 124 and the exterior of the device. Between the bellows 117 and the housing 110 is provided an actuator stack 111. A control unit 150 drives the actuator stack 111 via control cable 151.

Due to its strength and rigidity, metal is suitable material for housing 111 in this embodiment (and in the embodiment of FIG. 3 as well). Where it is desirable to provide energy to the control unit 150 or to communicate with the control unit 150 in a wireless fashion as described further below, an opening may be provided in the metal housing 110 as illustrated in FIG. 4A, to address the shielding effects of the metal housing. Alternatively, the pump can be provided, for example, with an exterior coil (e.g., for transdermal energy coupling) and/or an exterior antenna (e.g., for communication), with electrical feed-throughs in the housing to connect the coil and/or antenna with the control unit.

FIG. 4B provides a detailed schematic cross-sectional view of area A, which is defined by the dashed lines of FIG. 4A. Referring now to FIG. 4B, a stack of counter-electrode layers 118, active layers 112 and electrolyte-containing layers 114 are shown.

As above, the counter-electrode layers 118 may be formed from a suitable electrical conductor, for example, a metal such as gold or platinum. The electrolyte within the electrolyte-containing layers 114 can be, for example, a liquid, a gel, or a solid, with appropriate measures being taken, where needed, to prevent short-circuiting between the counter-electrodes 118 and the active layers 112. The active layer 112 comprises an electroactive polymer, for example, polypyrrole, polysulfone, polyacetylene or polyaniline. The actively layers 112 can also be optionally be provided with conductive electrical contacts (not shown), if desired, to enhance electrical contact with the control unit.

During operation, an appropriate potential difference is applied across the active layers 112 and the counter-electrode layers 118 using control unit 150. In certain embodiments, all of the active layers 112 are shorted to one another, as are all of the counter-electrode layers 118, allowing the active layers 112 to expand and contract simultaneously. As above, the electroactive polymer active layers 112 expand and contract upon establishing an appropriate potential difference between the active layers 112 and the counter-electrode layers 118. This, in turn, expands and contracts the actuator stack 111.

Upon expansion of the actuator stack 111, the bellows 117 are compressed, pressurizing the medication within medication reservoir 124. Contraction of the actuator stack 111, on the other hand, permits the medication reservoir 124 to be recharged with medication.

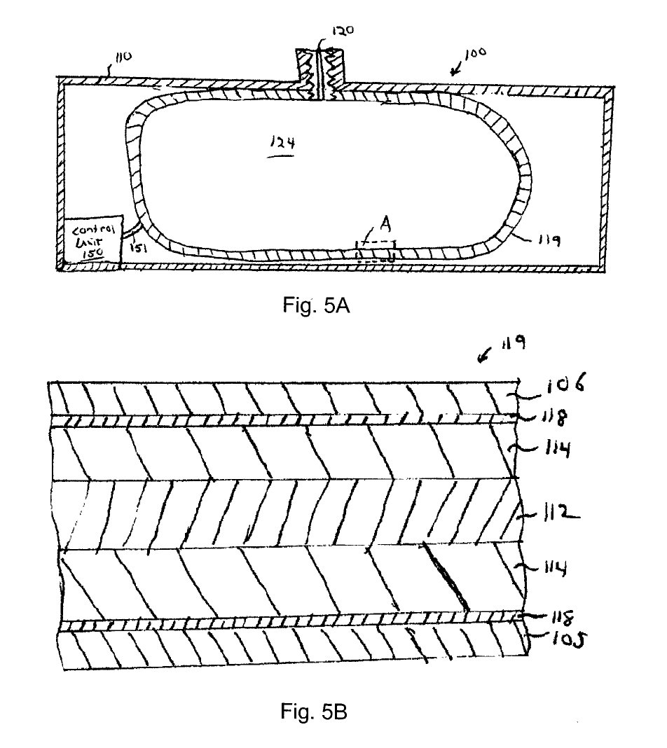

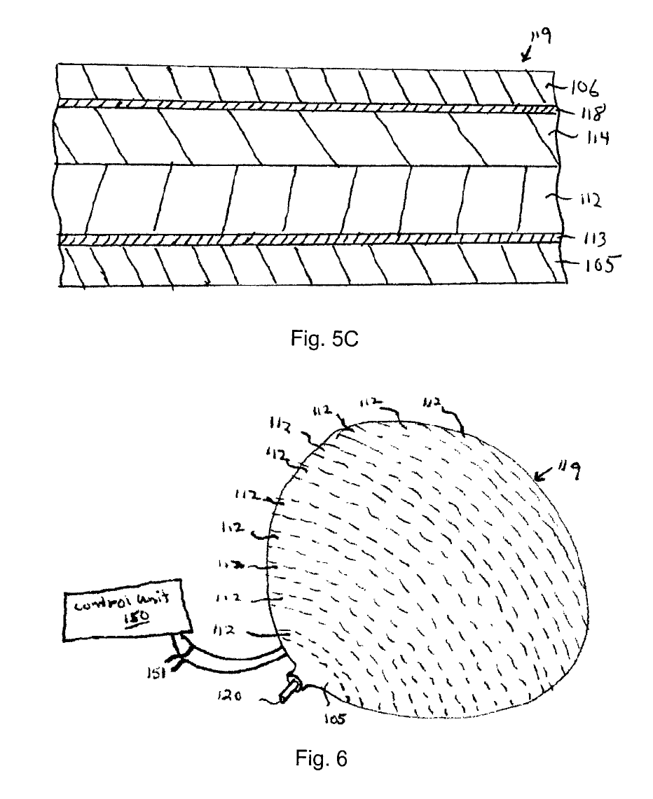

An infusion pump in accordance with yet another embodiment of the present invention is illustrated in FIG. 5A. In this embodiment, the infusion pump 100 contains an expandable enclosure such as a bladder 119, the interior of which defines a medication reservoir 124. An outlet port 120 provides fluid communication between the medication reservoir 124 and the exterior of the pump 100. A control unit 150 drives electroactive polymer actuators disposed within the wall of bladder 119 via control cable 151. By applying an appropriate potential, control unit 150 can either contract the bladder 119, for example, to force medication from the medication reservoir 124 through the outlet port 120, or expand the bladder 119, for example, to allow the medication reservoir 124 to be refilled with medication. Because the pumping action does not require the exertion of force on the housing 110, the walls of the housing 110 can be lighter (e.g., allowing a dense material such as metal to be replaced with a less dense material such as a polymeric material) and/or thinner, which reduces the size and weight of the pump. Indeed, in some embodiments, the housing 110 can be dispensed with entirely, as discussed below.

FIG. 5B provides a detailed schematic cross-sectional view of area A, which is defined by the dashed lines in FIG. 5A. Referring now to FIG. 5B, a layer stack is illustrated which includes an outer layer 105, an inner layer 106, an active layer 112, counter-electrode layers 118 and electrolyte-containing layers 114.

As above, the counter-electrode layers 118 can be formed from any suitable electrical conductor, for example, a metal such as gold or platinum. The counter-electrode 118 can be, for example, in wire or film form and can be applied, for example, by electroplating, chemical deposition, or printing. The electrolyte within the electrolyte-containing layers 114 can be based, for example, a liquid, gel, or solid electrolyte, with appropriate measures being taken where needed to prevent short-circuiting between the counter-electrode layers 118 and the active layer 112.

The active layer 112 comprises an electroactive polymer, for example, polypyrrole, polysulfone, polyacetylene or polyaniline. Moreover, the actively layer 112 can optionally be provided with a conductive electrical contact (not shown), if desired, to enhance electrical connection with the control unit.

The outer and inner layers 105, 106 can be selected from a number of flexible materials, and can be formed, for example, from one or more polymeric materials. Polymeric materials useful in the construction of the outer and inner layers 105, 106 include the following polymeric materials: polyolefins such as metallocene catalyzed polyethylenes, polypropylenes, and polybutylenes and copolymers thereof; ethylenic polymers such as polystyrene; ethylenic copolymers such as ethylene vinyl acetate (EVA), butadiene-styrene copolymers and copolymers of ethylene with acrylic acid or methacrylic acid; polyacetals; chloropolymers such as polyvinylchloride (PVC); fluoropolymers such as polytetrafluoroethylene (PTFE); polyesters such as polyethylene terephthalate (PET); polyester-ethers; polysulfones; polyamides such as nylon 6 and nylon 6,6; polyamide ethers such as polyether block amides; polyethers; elastomers such as elastomeric polyurethanes and polyurethane copolymers; silicones; polycarbonates; polychloroprene; nitrile rubber; butyl rubber; polysulfide rubber; cis-1,4-polyisoprene; ethylene propylene terpolymers; as well as mixtures and block or random copolymers of any of the foregoing are examples of polymers useful for manufacturing the medical devices of the present invention. In certain embodiments, the outer and inner layers 105, 106 are formed from elastomeric polymeric materials.

In general, the inner layer 106 is compatible with the medication in the medication reservoir 124. Where the outer layer 105 contacts bodily tissue (e.g., where no external housing is utilized), the outer layer is typically both biostable and biocompatible.

As a specific example, the outer and inner layers 105, 106 can comprise urethane or silicone polymers, the counter-electrode layers 118 can comprise a thinly deposited layer of gold (which can be, for example, in the form a foil or of printed wiring), the active layer 112 can comprise polypyrrole, and the electrolyte-containing layers can comprise a gel (e.g., PMMA with salt dopant).

During operation, control unit 150 is used to apply a potential difference across the active layer 112 and the counter-electrode layers 118 as previously discussed. This results in the passage of current between the active layer 112 and the counter-electrode layers 118, resulting in the contraction or expansion of active layer 112. In certain embodiments, all of the active layers 112 are shorted to one another, as are all of the counter-electrode layers 118.

FIG. 5C is an alternative design for the layer stack illustrated in FIG. 5B. Similar to FIG. 5B, FIG. 5C illustrates an outer layer 105, an inner layer 106, a counter-electrode layer 118, an electrolyte-containing layer 114, and an active layer 112. However, in FIG. 5C there is only a single electrolyte-containing layer and a single counter electrode 118 in the cross-section shown. FIG. 5C further includes a conductive electrical contact layer 113 for providing effective electrical connection with the active layer 112.

In some embodiments, the active layer 112 corresponds to one of a series of bands or fibers, which are wrapped around the bladder 119 in a fashion that is dependent upon the bladder geometry. For example, as can be seen in FIG. 6, a spherical bladder 119 can be encircled by a number of active layer bands 112, in a fashion analogous to lines of constant latitude on a globe. The volume of the bladder 119 is reduced upon contraction of the active layer 112 bands or fibers, forcing medication from the pump 100. While a spherical geometry is illustrated, other geometries can be used, including elliptical and cylindrical geometries. Note that the bladder 119 and the control unit 150 in FIG. 6 are provided independent of any housing.

Layered structures are efficient from a manufacturing perspective. Using the structure of FIG. 5B as a specific example, the outer layer 105 can be used as a substrate layer, with the following layers formed over the outer layer 105 in sequence: first counter-electrode layer 118, first electrolyte-containing layer 114, active layer 112, second electrolyte-containing layer 114, second counter-electrode layer 118 and inner layer 106.

Using the structure of FIG. 5C as another specific example, a first structure can be formed by depositing counter-electrode layer 118 on inner layer 106 (thus using layer 106 as a substrate layer). Similarly, a second structure can be formed by depositing contact layer 113 on outer layer 105 (thus using layer 105 as a substrate layer), followed by deposition of active layer 112. An electrolyte layer 114 can subsequently be laminated between these two structures.

Myriad additional configurations are possible. For example, a counter-electrode, or a series of counter-electrodes (as well as associated wiring for interconnection purposes), can be deposited on a first substrate layer. An electroactive polymer region, or a series of electroactive polymer regions (as well as associated contact wiring for interconnection purposes, if desired) can be deposited on a second substrate layer. Further, if desired, a series of strain gauges (see below) and associated interconnect wiring can be deposited on a third substrate layer. These layers can then be laminated, along with an electrolyte-containing layer. In this case, each substrate layer is similar to a flexible printed circuit board in that the elements are printed upon a flexible substrate. Moreover, as an alternative to providing each substrate layer with its own interconnect wiring, a separate interconnect layer can be provided on a single substrate, with appropriate connections to other substrate layers being made, for example, by means of plated through-holes or vias (these also can function as “rivets” to hold the stack together).

Still other alternative embodiments are clearly possible in addition to the laminated structures discussed above. For example, prefabricated electroactive polymer actuators (e.g., the actuator of FIG. 2) and associated control cables can be woven or otherwise incorporated into the layers of the elastic bladder wall.

Various liquid medications (also referred to herein using terms such as “therapeutic agents” and “drugs”) can be infused using the pumps of the present invention. Specific examples include the infusion of insulin for the treatment of diabetes, opiate infusion for use in patient analgesia, local infusion of drugs for cancer chemotherapy, infusion of stimulants for the treatment of heart failure or arrhythmia, infusion of drugs for seizure treatment, and so forth. Many additional medication/condition combinations are known in the art.

Medication can be targeted for systemic delivery or for delivery to a local site of interest. For example, for systemic delivery, medicine can be directed through a catheter and into the portal vein at a position downstream from liver, avoiding hepatic clearance issues. As examples of local delivery, medicine can be directed through a catheter into the arterial side of the vascular system that supplies a specific region (e.g., for the treatment of a tumor), into the spinal fluid (e.g., for epidural treatment of pain), and so forth. Numerous other delivery arrangements are known in the art and can be used in connection with the present invention.

In some cases, the volume of the medication reservoir can be inferred from the intrinsic position-dependent electrical properties of the electroactive polymer actuators. However, a number of strain gauges can be employed to provide electronic feedback concerning reservoir volume or pressure. This electronic feedback will also provide a number of additional advantages, including compensation for physiologic changes, greater stability, error correction, and immunity from drift. Strain gauges suitable for use in the present invention include (a) feedback electroactive polymer elements whose impedance or resistance varies as a function of the amount of strain in the device, (b) linear displacement transducers (e.g., an iron slug slidably positioned in the core of a coil) and (c) conventional strain gauges in which the resistance of the device varies as a function of the amount of strain in the device, thus allowing the amount of strain to be readily quantified and monitored. Such strain gauges are commercially available from a number of different sources, including National Instruments Co., Austin, Tex., and include piezoresistive strain gauges (for which resistance varies nonlinearly with strain) and bonded metallic strain gauges (for which resistance typically varies linearly with strain).

The volume of the dispensed medication is equal to the volumetric change of the medication reservoir. Flow rate can be calculated based on volumetric change as a function of time.

The control unit 150 used in connection with the infusion pumps of the present invention is typically provided with a power unit. The power unit can include one or more batteries, which may be rechargeable, for example, using a wireless power transmission interface. An example of a wireless power transmission interface is one based on transcutaneous induction of electromagnetic fields within an implanted coil, which is connected to the batteries in the pump. Recharging schemes of this type are presently used in connection with various implantable devices, including pacemakers and implantable defibrillators. Further information can be found, for example, in U.S. Pat. No. 5,954,058 and the references disclosed therein, which are hereby incorporated by reference.

The control unit is also preferably provided with a mechanism for supplying an appropriate control signals to the actuator(s), and any other control devices (e.g., control valves), within the infusion pumps of the present invention. As a specific example, control signals can be supplied to the actuator(s) by simply providing a subcutaneous switch, which can be operated by the patient or physician. The switch can be designed to apply a potential of first polarity from the battery to contract the actuators and deliver medication, and to apply a potential of opposite polarity from the battery to expand the actuators and allow the reservoir to be refilled with medication.

Control signals for the infusion pumps of the present invention can be generated based on a number of criteria. For instance, control signals can be generated based on time. Examples include delivery of medication based on a simple timer within the control unit, as well as delivery of medication at scheduled times and in scheduled dosages based on data that is stored to memory within the control unit.

Control signals can also be generated based on sensor feedback. For example, medication can be delivered using computation and servomechanism actuator control, based on sensors and automatic control algorithms (e.g., using a sensor and set-point algorithm). Sensors include physiological sensors (e.g., glucose sensors, O₂ sensors, or sensors for sensing other physiological fluid components), as well as sensors indicating the status of the pump (e.g., strain gauges providing feedback regarding reservoir volume). Information from the sensors can then be transported via lead or wireless link to the controller.

Control signals also can be generated based on based on external commands, including both hard-wired and wireless commands. For example, the patient can voluntarily increase dose as needed to manage pain within preprogrammed safety limits. In certain embodiments, control signals can be generated on patient demand by using a simple subcutaneous switch as discussed above. In certain other embodiments, control signals can be transmitted to the pump based on communication from an external electronic appliance, carrying out, for example, patient or caregiver instructions. Examples of such external electronic devices include stand-alone electronic devices (e.g., personal computers and personal digital assistants or “pdas”), an electronic device connected to a network, or an electronic device connected to the Internet.

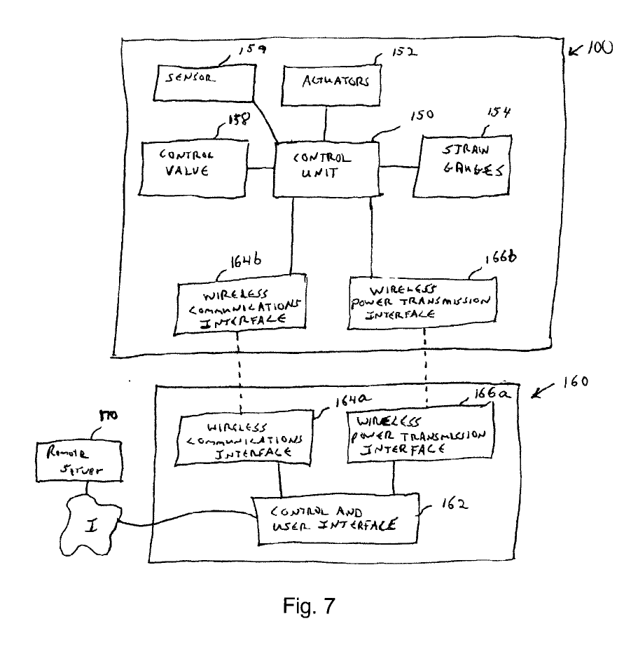

FIG. 7 is a simplified electrical schematic diagram of one infusion pump apparatus in accordance with an embodiment of the present invention. The apparatus includes infusion pump 100 and an associated external device (e.g., a personal computer 160). As previously discussed, the infusion pump 100 contains one or more electroactive polymer actuators 152. The infusion pump illustrated in FIG. 7 also includes one or more control valves 158, one or more strain gauges 154 and one or more sensors 159 (for example, a glucose senor, which allows, for example, for closed-loop control based on sensor input). A control unit 150, for example a computer equipped with an electronic interface and drivers, (a) provides an appropriate signal to expand or contract the actuators as required, (b) provides an appropriate signal to open or close the control valve as required, and (c) collects information from the strain gauges 154 and sensor 159 (e.g., by measuring impedance and/or voltage). Control unit 150 is also provided with a source of power, typically one or more batteries.

Exterior programming and control of the pump 100 is implemented in FIG. 7 via computer 160, which contains components for control and user interface 162. Data is exchanged between the computer 160 and the pump 100 via a wireless communication interface 164a, 164b. Inexpensive wireless interfaces are presently available from a number of sources, including Bluetooth™ wireless interfaces available from Motorola and IEEE 802.11b wireless interfaces available, for example, from Cisco, Apple and Lucent. The wireless interface 164a within the computer 160 communicates with a companion wireless interface 164b within the infusion pump 100. Power is directed to the pump 100 via a wireless power transmission interface 166a, 166b, which can be based on transcutaneous induction of electromagnetic fields within an implanted coil as previously discussed. In the embodiment illustrated, the computer 160 is equipped to communicate with a remote server 170 via the Internet I.

Although the present invention has been described with respect to several exemplary embodiments, there are many other variations of the above-described embodiments that will be apparent to those skilled in the art, even where elements have not explicitly been designated as exemplary. It is understood that these modifications are within the teaching of the present invention, which is to be limited only by the claims appended hereto.

What is claimed is:

1. A drug delivery pump apparatus comprising:
(a) a contractible and expandable enclosure having an interior volume defining a medication reservoir;
(b) an electroactive polymer actuator, said electroactive polymer actuator reducing said interior volume of said contractible and expandable enclosure based upon received control signals;
(c) a medication outlet port providing fluid communication between said interior volume of said contractible and expandable enclosure and an exterior of said delivery pump apparatus; and
(d) a control unit electrically coupled to said actuator and sending said control signals to said actuator.

2. The drug delivery pump apparatus of claim 1, wherein said contractible and expandable enclosure comprises two or more electroactive polymer actuators.

3. The drug delivery pump apparatus of claim 1, wherein said interior volume of said contractible and expandable enclosure is reduced upon expansion of said electroactive polymer actuator.

4. The drug delivery pump apparatus of claim 1, wherein said interior volume of said contractible and expandable enclosure is reduced upon contraction of said electroactive polymer actuator.

5. The drug delivery pump apparatus of claim 1, further comprising a housing that encloses said contractible and expandable enclosure.

6. The drug delivery pump apparatus of claim 5, wherein said housing further encloses said control unit.

7. The drug delivery pump apparatus of claim 1, wherein said contractible and expandable enclosure comprises a bellows.

8. The drug delivery pump apparatus of claim 7, wherein said bellows are compressed upon the expansion of said actuator.

9. The drug delivery pump apparatus of claim 1, wherein said actuator comprises an electroactive polymer region, a counter-electrode region, and an electrolyte-containing region disposed between said electroactive polymer region and said counter-electrode region.

10. The drug delivery pump apparatus of claim 9, wherein said electroactive polymer comprises an electroactive polymer selected from polyaniline, polysulfone, and polyacetylene.

11. The drug delivery pump apparatus of claim 9, wherein said electroactive polymer comprises polypyrrole.

12. The drug delivery pump apparatus of claim 9, further comprising a conductive housing that encloses said contractible and expandable enclosure, wherein said housing serves as said counter-electrode or as a contact for said electroactive polymer.

13. The drug delivery pump apparatus of claim 9, wherein said contractible and expandable enclosure comprises a conductive bellows and wherein said bellows further serves as said counter-electrode or as a contact for said electroactive polymer.

14. The drug delivery pump apparatus of claim 1, wherein said interior volume of said contractible and expandable enclosure is reduced upon expansion of an actuator stack that comprises a plurality of electroactive polymer layers, a plurality of counter-electrode layers, and a plurality of electrolyte-containing layers.

15. The drug delivery pump apparatus of claim 1, wherein said contractible and expandable enclosure comprises an elastic wall.

16. The drug delivery pump apparatus of claim 1, wherein said actuator is disposed within or upon a wall of said contractible and expandable enclosure.

17. The drug delivery pump apparatus of claim 16, wherein said enclosure wall comprises an inner layer, an outer layer, a counter-electrode region, an electrolyte-containing region and a electroactive polymer region, and wherein said counter-electrode region, said electrolyte-containing region and said electroactive polymer region are disposed between said inner and outer layers.

18. The drug delivery pump apparatus of claim 1, wherein said medication outlet port is provided with a control valve that is operable based upon received control signals.

19. The drug delivery pump apparatus of claim 1, further comprising a wireless power transmission interface coupled to a rechargeable battery within said control unit.

20. The drug delivery pump apparatus of claim 1, further comprising a first wireless transceiver coupled to said control unit.

21. The drug delivery pump apparatus of claim 1, further comprising a sensor coupled to said control unit.

22. The drug delivery pump apparatus of claim 21, wherein said sensor is a strain gauge.

23. The drug delivery pump apparatus of claim 21, wherein said sensor is a chemical sensor that measures a detectable chemical species.

24. The drug delivery pump apparatus of claim 5, wherein said electroactive polymer actuator is disposed between said housing and said contractible and expandable enclosure, and wherein said interior volume of said contractible and expandable enclosure is reduced upon expansion of said electroactive polymer actuator.

25. A method of delivering a liquid therapeutic agent to a patient comprising:
providing the infusion pump apparatus of claim 1;
placing said outlet port in fluid communication with a patient; and
sending said control signals to said actuator to reduce said internal volume of said contractible and expandable enclosure and force a portion of the liquid therapeutic agent within said medication reservoir through said outlet port and into said patient.

26. The method of claim 25, wherein said infusion pump apparatus is implanted or inserted within said patient.

27. The method of claim 25, wherein said control signals are generated based upon a user-activatable switch.

28. The method of claim 27, wherein said user-activatable switch is inserted or implanted within said patient.

29. The method of claim 25, wherein said control signals are generated based on the passage of a predetermined interval of time.

30. The method of claim 25, wherein said control signals are generated based upon input from a chemical sensor that measures a detectable chemical species.
